(19) United States
(12) Patent Application Publication
(10) Pub. No.: US 2024/0368710 A1
(43) Pub. Date: Nov. 7, 2024

(54) STREAMLINED PLATFORM FOR BACTERIAL IDENTIFICATION AND ANTIBIOTIC SUSCEPTIBILITY TEST

(71) Applicant: THE JOHNS HOPKINS UNIVERSITY, Baltimore, MD (US)

(72) Inventors: Tza-Huei Jeff WANG, Germantown, MD (US); Pornpat ATHAMANOLAP, Baltimore, MD (US); Liben CHEN, Baltimore, MD (US); Kuangwen HSIEH, Baltimore, MD (US)

(21) Appl. No.: 18/772,429

(22) Filed: Jul. 15, 2024

Related U.S. Application Data
(62) Division of application No. 16/491,022, filed on Sep. 4, 2019.
(60) Provisional application No. 62/467,482, filed on Mar. 6, 2017.

(51) Int. Cl. C12Q 1/689; B01L 3/00; C12Q 1/06; C12Q 1/6851; C12Q 1/686

(52) U.S. Cl. CPC C12Q 1/689; B01L 3/502707; B01L 3/502761; C12Q 1/06; C12Q 1/6851; C12Q 1/686; B01L 2200/0668; B01L 2300/0864; B01L 2300/0887; B01L 2300/0893; B01L 2300/0896; B01L 2400/0655; C12Q 2600/156

(57) ABSTRACT

Described are methods for identifying antibiotic resistant bacteria, quantifying bacteria growth, and applying an antibiotic susceptibility test (AST) in one or more biological samples containing a bacteria and chips used in these methods.

### REFERENCE TO RELATED APPLICATIONS

This application is a division of U.S. patent application Ser. No. 16/491,022, filed on Sep. 4, 2019, now pending, which is a national stage entry from International Application No. PCT/US2018/021031, filed on Mar. 6, 2018, published as International Publication No. WO2018165080A1 on Sep. 13, 2018, which claims the benefit of U.S. Provisional Patent application 62/467,482, filed Mar. 6, 2017, the contents of which are hereby incorporated by reference herein in their entireties.

### STATEMENT OF GOVERNMENTAL INTEREST

This invention was made with government support under grant no. R01AI117032 awarded by the National Institutes of Health. The government has certain rights in the invention.

### BACKGROUND OF THE INVENTION

For managing dangerous infections, clinicians need to know what bacteria strain(s) is/are causing the infections, the bacterial loads, and the antibiotic resistance profile of the bacteria. Without knowing this vital information in a timely manner, it is impossible to begin using narrow-spectrum antibiotics that can effectively treat the patients. Importantly, these 3 aspects are all important, knowing one aspect earlier does not help. For example, simply knowing the species of the bacteria without knowing the antibiotic resistance can lead to use of ineffective antibiotics (e.g., ESBL enterobacteriaceae). It is thus evident that integrated and streamlined methods that can: 1) identify infectious bacteria (ID), 2) quantify bacterial load, and 3) perform antibiotic susceptibility tests (AST) directly from clinical samples hold the key to effective management of infectious diseases. Recent advances in molecular and microfluidic technologies have enabled development of increasingly effective strategies toward addressing this unmet need. However, research efforts to date have almost exclusively taken a “discrete” approach, providing only partial solution to the problem. A complete solution capable of supplanting blood culture has yet to be realized.

### SUMMARY OF THE INVENTION

One embodiment of the present invention is a method for identifying and quantifying the growth of antibiotic resistant bacteria comprising the steps of: obtaining a biological sample of a subject comprising unidentified bacteria; culturing the unidentified bacteria in a first broth comprising an antibiotic; culturing the unidentified bacteria in a second broth substantially free of the antibiotic; amplifying DNA of the unidentified bacteria in the first and second broth using polymerase chain reaction (PCR) forming amplified DNA that is quantified and correlates with the unidentified bacteria growth; identifying the antibiotic sensitivity or resistance of the unidentified bacteria by comparing the bacteria growth in the first broth with the bacteria growth in the second broth; and identifying the species of unidentified bacteria by determining a first melting curve of the unidentified bacteria and comparing it to one or more melting curves of known bacteria stored in a computer wherein a positive identification occurs when the first melting curve of the unidentified bacteria is equivalent to the one or more melting curves of known bacteria stored in the computer (FIG. 1). In some embodiments the method may include a step of separating the bacteria from the biological sample. A suitable biological sample includes whole blood, plasma, serum, RBC fraction, urine, saliva, cerebrospinal fluid, semen, sweat, bile, gastric contents, breast milk, exudates, ascites, lymph, sputum, lavage fluid, bronchial fluid, or a combination thereof. The methods of the present invention may be modified based on the biological sample used. For example, if blood is the biological sample, the method may comprise a step of preferential lysis of blood cells but not the bacteria, followed by isolation of bacteria. The bacteria may be cultured in the range of 15 minutes to 24 hours, 1 hour to 20 hours, 2 hours to 18 hours, 4 hours to 15 hours, or 6 hours to 10 hours, depending upon the specific application. The step of identifying the species of unidentified bacteria is preferably performed using high-resolution melt curve analysis (HRMA) to generate the first melting curve of the unidentified bacteria. Suitable antibiotics used in the present invention include streptomycin, gentamicin, penicillin, ceftazidime, trimethoprim-sulfamethoxazole or a combination thereof, as examples. In some applications, polymerase chain reaction (PCR) comprises a fluorescent intercalating dye and the growth of bacteria is determined by identifying the amount of amplified DNA produced during PCR by generating a fluorescent signal and then generating a quantification cycle (Cq) correlated to bacteria growth. The method of determining when a bacteria is antibiotic resistant or sensitive is by comparing the difference in Cq between a bacteria grown in a broth having an antibiotic with the same bacteria grown in a broth without an antibiotic. The Cq number may vary depending upon the bacteria and the antibiotic used in the methods of the present invention resulting in the difference in Cq varying and the ultimate determination of antibiotic resistance or sensitivity varying for each particular bacteria grown in a particular antibiotic, for example. Generally, antibiotic resistance may be determined when the Cq differences is less than 1.7 or in the range of −0.2 to 2.0, 0.5 to 1.8, or 1.0 to 1.4. Generally, antibiotic sensitivity may be determined when the Cq differences is greater than 1.7, 1.5 to 10, 2 to 12, 3 to 7, or 1.8 to 4.

Another embodiment of the present invention is the use of digital polymerase chain reaction PCR (dPCR) for identifying and quantifying the growth of one or more species of bacteria in the sample or “target” (FIG. 2). Following bacteria culture and lysis, the extracted bacterial DNA and PCR assay, which comprises of a fluorescent intercalation dye, are mixed into a very large number of separate small volume reactions so that there is either zero or one bacterial DNA present in any individual reaction (Sykes P J, Nech S H, et al. (1992). Quantitation of targets for PCR by use of limiting dilution. BioTechniques. 13:444-449; and Vogelstein B and Kinzler K W. (1999) Digital PCR. Proc Natl Acad Sci USA. 96:9236-9241). After the completion of reactions, any target-containing compartments will become brightly fluorescent while compartments without targets will have only background fluorescence. The step of identifying the species of unidentified bacteria is preferably performed using digital high-resolution melt curve analysis (dHRMA) in each positive reaction to generate the melting curve of the unidentified bacteria. A positive identification occurs when the first melting curve of the amplified DNA in the well is equivalent to the one or more melting curves of the PCR amplicons stored in the computer. Subsequently, the growth of each of the identified bacteria species is determined by the amount of nucleic acids of the identified bacteria species calculated by the number of the wells of the positive identification associated to the identified bacteria species. The method of determining when a bacteria is antibiotic resistant or sensitive is by comparing the difference of the amount of nucleic acids of the bacteria grown in a broth having an antibiotic with the same bacteria grown in a broth without an antibiotic. The amount of nucleic acids may vary depending upon the bacteria and the antibiotic used in the methods of the present invention, which also results in varying the ultimate determination of antibiotic resistance or sensitivity for each particular bacteria grown in a particular antibiotic. For example, when performing dPCR using a 96-well plate antibiotic resistance may be determined when the difference of the amount of nucleic acids of the bacteria derived from the first broth and the second broth is less than 0.2 fold of the amount of nucleic acids of bacteria derived from the second broth. Generally, antibiotic sensitivity may be determined when the difference of the amount of nucleic acids of the bacteria derived from the first broth and the second broth is greater than 0.2 fold of the amount of nucleic acids of bacteria derived from the second broth.

Another embodiment of the present invention is the use of an array of digital reaction chambers for identifying and quantifying the growth of bacteria at the single-cell resolution (FIG. 3). In the digital (single-cell) assay, the bacteria sample, culture medium and PCR assay, which comprises of a fluorescent intercalation dye, are mixed into a very large number of separate small volume reactions so that there is either zero or one bacteria present in any individual reaction. After the completion of culture and PCR reactions, any bacteria-containing compartments will become brightly fluorescent while compartments without bacteria will have only background fluorescence. The step of identifying the species of unidentified bacteria is preferably performed using high-resolution melt curve analysis (HRMA) in each “positive reaction” to generate the melting curve of the unidentified bacteria. A positive identification occurs when the first melting curve of the amplified DNA is equivalent to the one or more melting curves of the PCR amplicons stored in the computer. The growth of each of the identified bacteria species is determined by the average quantification cycle (Cq) derived from the wells of the positive identification associated to the identified bacteria species. The method of determining when a bacteria is antibiotic resistant or sensitive is by comparing the difference in average Cq between each bacteria species grown in a broth having an antibiotic with the same bacteria species grown in a broth without an antibiotic. The Cq number may vary depending upon the bacteria and the antibiotic used in the methods of the present invention, which also results in varying the ultimate determination of antibiotic resistance or sensitivity for each particular bacteria grown in a particular antibiotic, for example. Generally, antibiotic resistance may be determined when the differences of average Cq of the bacteria derived from the first broth and the second broth are less than 1.2. Generally, antibiotic sensitivity may be determined when differences of average Cq between the bacteria derived from the first broth and the second broth are greater than 1.2.

Another embodiment of the present invention is a chip for identifying bacteria, quantifying growth, AST, and/or antibiotic resistant or sensitive, bacteria comprising: a flow chamber comprising one or more flow channels, microvalves, and picowells wherein the flow channels are in contact with the microvalves and the picowells, the microvalves are located adjacent to the picowells and are capable of being pressurized, and when the microvalves are pressurized they enclose a segment of the flow channel and a picowell forming a digital reaction chamber. The chips of the present invention may be of varying size depending upon the application; for example, in some embodiment a flow chamber is a microfluidic flow chamber and the picowells each comprise a predetermined amount of antibiotic ranging from 0.001 μg/mL to 256 μg/mL of antibiotic. FIG. 9 illustrates the dimensions of one embodiment of a chip of the present invention. The picowells dimensions may vary depending upon the application and maybe in the range of 50 pL to 50 nL, 100 pL to 10 nL, or 1000 pL to 5 nL, for example. The flow channels dimensions may vary depending upon the application and be in the range of 10 μm to 1000 μm, 50 μm to 750 μm, or 100 μm to 500 μm, for example. The chips of the present invention may be pre-coated with an antibiotic prior to sample loading. In such chip embodiments, at least 10%, at least 40%, or at least 50% of the picowells comprise an antibiotic. In one embodiment of the chip, the microvalves and the picowells alternate on the flow channels wherein the reaction chambers are located between the microvalves. Preferably, the microvalves are designed to become pressurized and expand and enclose the channels forming digital reaction chambers. Other embodiments include chips made of gas permeable material such as a silicone material such as polydimethylsiloxane (PDMS), for example, and/or the picowells are covered with a gas-impermeable material, such as glass, to make them impermeable to gas.

Another embodiment of the present invention is a system for identifying antibiotic resistant or sensitive bacteria comprising: a chip of the present invention; and a digital analysis system able to quantify DNA and identify strains of bacteria wherein the digital analysis system includes a thermal cycler for performing PCR and HRMA, an optical setup for monitoring fluorescent level and a computer for obtaining data and analyzing results that comprises stored melting curves of bacteria and is able to compare melting curves of bacteria. The system may include an external pump able to drive continuous, unidirectional, or bidirectional fluid through the flow channels in one or more passes. Most fluids may be suitable for use in the present invention including air water, biological samples in liquid form, as examples.

Another embodiment of the present invention is a method for identifying antibiotic resistant or sensitive bacteria comprising the steps of: a) providing a chip comprising a flow chamber comprising one or more flow channels; microvalves, and picowells, wherein the flow channels are in contact with the microvalves and the picowells, the microvalves are located adjacent to the picowells and are capable of being pressurized, and when the microvalves are pressurized they enclose a segment of the flow channel and a picowell forming a digital reaction chamber; b) loading a biological sample comprising bacteria with an antibiotic on to the chip through the one or more flow channels wherein each picowell contains either 0 to 1 bacterial cell; c) injecting fluid into the one or more flow channels but not the picowells; d) culturing the bacteria in the picowells; c) loading a polymerase chain reaction (PCR) mixture on to the chip through the one or more flow channels; f) pressurizing the microvalves enclosing the chambers forming a digital reaction chamber comprising the PCR mixture; g) amplifying DNA of the bacteria using polymerase chain reaction (PCR) forming amplified DNA that is quantified and correlates with the bacteria growth; and h) identify the antibiotic sensitivity or resistance of the unidentified bacteria by comparing the bacteria growth to a reference bacteria growth. The method may include additional steps: i) performing melt curve analysis of the amplified DNA; and j) identifying the strain of the bacteria by determining a first melting curve of the bacteria and comparing it to melting curves of one or more known bacteria stored in a computer wherein a positive identification occurs when the first melting curve of the bacteria is equivalent to a second melting curve of a known bacteria. Another additional step maybe lysing the bacteria with a buffer able to lyse the bacteria. The preferred reference bacteria growth is obtained from the biological sample comprising the bacteria without an antibiotic that has undergone the method. The methods of the present invention may performing polymerase chain reaction (PCR) using digital PCR and/or perform melt curve analysis utilizes digital HRMA (dHRMA).

One embodiment of the present invention is a digital method for identifying and quantifying the growth of antibiotic resistant or sensitive bacteria comprising the steps of: obtaining a biological sample of a subject comprising unidentified bacteria; culturing the unidentified bacteria in a first broth comprising an antibiotic; culturing the unidentified bacteria in a second broth substantially free of the antibiotic; lysing the unidentified bacteria in the first and second broth to release bacteria nucleic acid; diluting the bacteria nucleic acids of unidentified bacteria in first and the second broth and then placing them in two separate arrays of wells so that each well contains no more than one copy of the bacteria nucleic acid; amplifying the bacteria nucleic acid in each well with a mixture including a fluorescent intercalating dye using polymerase chain reaction (PCR) forming amplified DNA; identifying a species of the unidentified bacteria or an identified bacteria species by determining a first melting curve of the amplified DNA in each well and comparing it to one or more melting curves of PCR amplicons of known bacteria stored in a computer wherein a positive identification occurs when the first melting curve of the amplified DNA in the well is equivalent to the one or more melting curves of the PCR amplicons stored in the computer; and identifying the growth of each of the identified bacteria species by determining the amount of bacteria nucleic acid of the identified bacteria species calculated by the number of the wells of the positive identification associated to the identified bacteria species; wherein an identified bacteria species is antibiotic resistant based on the difference of the amount of nucleic acids of the bacteria derived from the first broth and the second; and wherein an identified bacteria species is antibiotic sensitive when there is a difference of the amount of nucleic acids of the bacteria derived from the first broth and the second broth.

Another embodiment of the present invention is a method for identifying and quantifying the growth of antibiotic resistant or sensitive bacteria comprising the steps of: obtaining a biological sample of a subject comprising unidentified bacteria; mixing the unidentified bacteria in a first broth comprising an antibiotic; mixing the unidentified bacteria in a second broth substantially free of the antibiotic; diluting the unidentified bacteria in the first and second broth and place them in two separate arrays of wells so that each well contains no more than one bacteria; culturing the one bacteria placed in each well forming a colony of one bacteria; lysing the colony of one bacteria to release bacteria nucleic acid; amplifying the bacteria nucleic acid of the colony in each well with a mixture including a fluorescent intercalating dye using polymerase chain reaction (PCR) forming amplified DNA that is quantified and correlates with the unidentified bacteria growth; identifying the species of the unidentified bacteria in each well or an identified bacteria species by determining a first melting curve of the amplified DNA of the unidentified bacteria and comparing it to one or more melting curves of PCR amplicons of known bacteria stored in a computer wherein a positive identification occurs when the first melting curve of the amplified DNA is equivalent to the one or more melting curves of the PCR amplicons stored in the computer; and identifying the growth of each of the identified bacteria species by the average quantification cycle (Cq) derived from the wells of the positive identification associated to the identified bacteria species; wherein an identified bacteria species is antibiotic resistant when there is a difference of average Cq of the bacteria derived from the first broth and the second broth; and wherein an identified bacteria species is antibiotic sensitive when there is a difference of average Cq between the bacteria derived from the first broth and the second broth.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change (increase or decrease) in the growth of a bacteria as detected by methods such as those described herein. As used herein, an alteration includes a 10% change in expression levels, preferably a 25% change, more preferably a 40% change, and most preferably a 50% or greater change in expression levels.”

By “quantification cycle” or “Cq” is meant a measurement taken in a real time PCR assay whereby a positive reaction is detected by accumulation of a signal, such as a fluorescent signal. The Cq (quantification cycle) is defined as the number of cycles required for the signal to cross the threshold (ie. exceeds background level). Cq levels are inversely proportional to the amount of target nucleic acid in the sample (i.e. the lower the Cq level the greater the amount of target nucleic acid in the sample).

By “digital polymerase chain reaction” or “digital PCR, DigitalPCR, dPCR, or dePCR” is a biotechnological refinement of conventional polymerase chain reaction methods that can be used to directly quantify and clonally amplify nucleic acids strands including DNA, cDNA or RNA. The key difference between dPCR and traditional PCR lies in the method of measuring nucleic acids amounts, with the former being a more precise method than PCR. A “digital” measurement quantitatively and discretely measures a certain variable, whereas an “analog” measurement extrapolates certain measurements based on measured patterns. PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts. The method has been demonstrated as useful for studying variations in gene sequences-such as copy number variants and point mutations—and it is routinely used for clonal amplification of samples.

By “high resolution melt curve analysis” or “HRM or HRMA” is performed on nucleic acid, specifically double stranded DNA samples. The process is simply a precise warming of the DNA typically from around 50° C. up to around 95° C. At some point during this process, the melting temperature of the DNA is reached and the two strands of DNA separate or “melt” apart. The secret of HRM is to monitor this process happening in real-time. This is achieved by using a fluorescent dye. The dyes that are used for HRM are known as intercalating dyes and have a unique property. They bind specifically to double-stranded DNA and when they are bound they fluoresce brightly. In the absence of double stranded DNA they have nothing to bind to and they only fluoresce at a low level. At the beginning of the HRM analysis there is a high level of fluorescence in the sample because of the billions of copies of the DNA. But as the sample is heated up and the two strands of the DNA melt apart, presence of double stranded DNA decreases and thus fluorescence is reduced. The HRM machine has a camera that watches this process by measuring the fluorescence. The machine then simply plots this data as a graph known as a melt curve, showing the level of fluorescence vs the temperature.

By “melting curve analysis” is meant an assessment of the dissociation-characteristics of double-stranded DNA during heating. As the temperature is raised, the double strand begins to dissociate leading to a rise in the absorbance intensity, hyperchromicity. The temperature at which 50% of DNA is denatured is known as the melting point, though it is an inaccurate term as it has very little to do with a traditional melting point. The information gathered can be used to infer the presence and identity of single-nucleotide polymorphisms (SNP). This is because G-C base pairing have 3 hydrogen bonds between them while A-T base pairs have only 2. DNA with a higher G-C content, whether because of its source (G-C contents: E. coli 0.50, M. luteus 0.72, poly d (AT) 0.00) or, as previously mentioned, because of SNPs, will have a higher melting temperature than DNA with a higher A-T content. The information also gives vital clues to a molecule's mode of interaction with DNA. Molecules such as intercalators slot in between base pairs and interact through pi stacking. This has a stabilizing effect on DNA's structure which leads to a raise in its melting temperature. Likewise, increasing salt concentrations helps diffuse negative repulsions between the phosphates in the DNA's backbone. This also leads to a rise in the DNA's melting temperature. Conversely, pH can have a negative effect on DNA's stability which may lead to a lowering of its melting temperature.

By “sample” or “biological sample” is meant a sample including whole blood, plasma, scrum, RBC fraction, urine, saliva, cerebrospinal fluid, semen, sweat, bile, gastric contents, breast milk, exudates, ascites, lymph, sputum, lavage fluid, bronchial fluid, or a combination thereof.

By “reduces” is meant a negative alteration of at least 10%, 25%, 50%, 75%, or 100%.

A “reference” refers to a standard or control conditions such as a sample (for example a biological sample including blood containing a bacteria) that is a free, or substantially free, of an antibiotic. It is preferred that the reference has undergone a method of the present invention.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

FIG. 1 illustrates a protocol for combined ID (Identification of pathogen species) and AST (antibiotic susceptibility test) in bulk format. The protocol begins by dividing the sample into 2 portions; with and without antibiotic followed by brief incubation. Then, the cultured samples are added into PCR mixture to perform qPCR & HRMA. An additional step of DNA isolation may be performed prior to PCR. AST is determined based on Cq differences (ΔCq) and ID is obtained from HRMA based classification.

FIG. 2 illustrates a protocol for combined ID and AST using digital detection. The protocol begins by dividing the sample into 2 portions; with and without antibiotic followed by brief incubation. DNA isolation can be performed to extract bacterial DNA from the samples. Then, the samples are mixed with PCR mixture and diluted across very large number of isolated small volume reactions so that there is either zero or one bacterial DNA present in any individual reaction to perform digital PCR & digital HRMA. ID is obtained from HRMA based classification on each positive reactions. AST of each target bacterial species is determined by comparing the quantity of the DNA of the target bacteria from the 2 portions which indicates by the total number of positive identification associated to the identified target bacteria species.

FIG. 3 illustrates a protocol for combined ID and AST using single-cell based detection. The protocol begins by dividing the sample into 2 portions; with and without antibiotic. The samples are then diluted across multiple small reactions so that there is either zero or one bacterial cell present in any individual reaction for brief incubation followed by PCR & HRMA. A microfluidic device that is capable of reagents switching may be used. DNA extraction may be performed prior or with PCR step. ID is obtained from HRMA based classification on each positive reactions. Bacterial load is determined by counting the number of positive identifications (number of wells) of each bacterial species. AST is determined based on average Cq differences (ΔCq) between with and without antibiotic samples of each species.

FIG. 4 illustrates an example protocol of isolation of intact bacteria from blood through preferential lysis.

FIG. 5A-5B illustrates the comparison between the proposed AST approaches to conventional broth microdilutional method. (a) The plot shows Cq differences (ΔCq) of E. coli growing with different gentamicin dosage (0.125, 0.25, 0.5, 1, 2, 4 and 8 μg/mL) to no drug controls. Green area shows the standard MIC range of this E. coli sensitive strain (orange). ΔCq of E. coli resistant strain is shown in blue. (b) Conventional broth microdilutional method of the same drug concentration with overnight incubation.

FIG. 6A-6C illustrates an integrated broad-scale pathogen identification and growth detection via qPCR and HRMA. (a) Measurement of bacterial growth of E. coli and S. aureus under treatment of gentamicin (1 μg/mL) via qPCR detection of 16S rDNA. The growth status and antibiotic susceptibility of bacteria were determined by comparing the Cq value to the no-drug control. (b) Measured melt curves from amplicons of E. coli and S. aureus for both sensitive and resistant strands. (c) The species of the tested pathogens were correctly identified via a SVM algorithm and an archived melt curve database previously developed by our group.

FIG. 7A-7C illustrates digital detection using 96-well plate of a mixed sample between S. aureus and E. faecalis incubated with and without gentamicin 16 μg/mL. Orange lines represent S. aureus and blue lines represent E. faecalis. (a) Melt curves from the sample incubated without gentamicin (b) Melt curves from the sample incubated with gentamicin (c) AST can be determined using absolute count of each species which bases on the total number of positive reactions of each specific melt curve from HRMA.

FIG. 8A-8B illustrates a Microfluidic Dual-Digital Array (MD²A) chip for single-cell digital assay. (a) The proposed chip will use a facile approach to carry out the comprehensive digital assay. Step 1: Samples with bacteria and antibiotics is injected into the chip, where each picowell contains either 0 or 1 bacterial cell. Step 2: Air is injected into the device and flowed through the channels but not into the picowells, achieving effective digitization. Step 3: Singly isolated bacteria in picowells with pre-defined antibiotic conditions only require brief culturing before their growth (and hence antibiotic profile) can be determined. Step 4: A mixture of lysis buffer and PCR mix is injected into the device to displace air in the channel but not in the picowells, allowing reagent switching. Step 5: Microvalves are pressurized such that each microvalve encloses a segment of the channel adjacent to a picowell with sufficient PCR mix, thus forming a digital reaction chamber. Step 6: Quantitative real-time PCR (qPCR) and post-PCR high-resolution melt curve analysis (HRMA) are performed to identify bacteria, perform AST, and quantify initial bacterial load in the sample. (b) Conceptual demonstration of ID, AST, and quantification of polymicrobial infection enabled by the MD²A chip and assay. Three pathogens (E. coli, Klebsiella, and MRSA) are identified through melt curves and their quantity is determined by the number of positive PCR chambers. For each drug tested, the resistance profile for each pathogen is determined by the Cq of qPCR. The MD²A chip has the capacity to test multiple drugs in parallel.

FIG. 9A-9C illustrates a prototype digital bacteria picoarray chip with 4 units can test 4 conditions in parallel. For each unit, inlet/outlet branching channels connect an array of 960 picowells.

FIG. 10 illustrates sample loading, digitization, and reagent exchange microfluidic array devices with similar dimensions. Samples (presented by yellow dye) are effectively injected via pressure and digitized by air. Subsequently, reagents (represented by the blue dye) can be easily injected into the device without flowing into picowells, as they are occupied by samples. However, the small dimensions facilitate rapid diffusion and reagent exchange (˜40 s).

FIG. 11 illustrates a PCR of single S. aureus cell is reliably achieved in chambers that are picoliters in volume. Here, each strongly fluorescent chamber represents a successful PCR from a single S. aureus cell.

FIG. 12 illustrates a protocol for device fabrication based on multi-layer soft lithography. Briefly, molds for the fluid layer and the microvalve layer will be microfabricated on separate silicon wafers via photolithography. Replicas of these molds are then generated using PDMS casting on the molds. The fluidic and microvalve layer replicas are then aligned and thermally bonded in an oven to generate a single chip. Holes are punched in this chip at input and output ports for making fluidic connections with the chip. Finally, both the top and bottom surfaces of the chip are bonded to coverglasses, readying the chip for experiments.

FIG. 13 illustrates a system and the instrumentation of the present invention. The system includes a wide-field, high-sensitivity, real-time fluorescence imaging device that can image the entire microfluidic chip (FIG. 9A) and a programmable heater. The imaging device is composed of an excitation source (e.g., LED), an appropriate emission filter, and a detector (e.g., a high resolution CMOS digital camera). The chip is placed on the surface of the programmable heater, which is responsible for thermal cycling for PCR and HRMA.

### DETAILED DESCRIPTION OF THE INVENTION

Despite the clear need and the research advances to date, there still exist a major technological gap for developing an integrated and streamlined method for identifying infectious bacteria, quantifying bacterial load, and performing antibiotic susceptibility tests (AST) directly from clinical samples. Specifically, methods that can identify and quantify bacteria with high specificity and high sensitivity are based on genetic detection, whereas methods that can perform accurate AST are predominantly based on bacterial growth. These methods are inherently incompatible because genetic detection entails extracting nucleic acids out of the bacteria, rendering downstream AST impossible. To fill this technological gap, in the present invention, a streamlined assay in which the inventors have flipped the order by coupling growth-based AST with highly specific and sensitive genetic detection, thereby achieving a seamless integrated process. In the present invention, bacteria within a sample are first cultivated for a short period in the absence and presence of different antibiotic substances. To detect growth or no growth—a hence antibiotic sensitivity or resistance—the bacterial DNA is amplified via real-time quantitative PCR (qPCR) by means of a pair of universal primer. An increase in the DNA amount in the presence of the tested drug shows that the bacteria are phenotypically resistant; conversely, no growth in the presence of a drug indicates susceptibility. Precise quantification of bacterial load is readily achieved because each bacterial cell is isolated and detected via qPCR (or digital PCR). Finally, bacteria can be effectively identified via high resolution melt-curve analysis (HRMA) of PCR amplified DNA, wherein distinct shapes of the melt curves offer effective means for bacteria identification. The method of the present invention can be carried out in both bulk and digital format as is described in this application.

### A Streamlined Pathogen Diagnostic Bulk Assay of the Present Invention Integrates ID and AST in a Single Process.

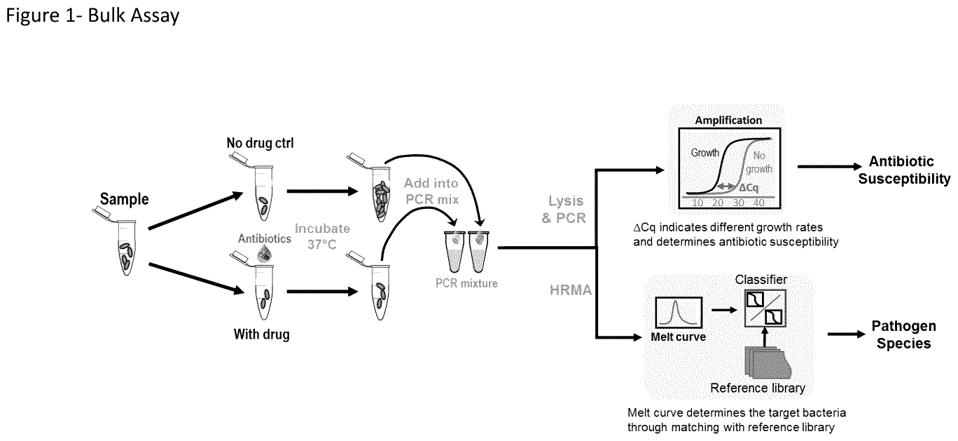

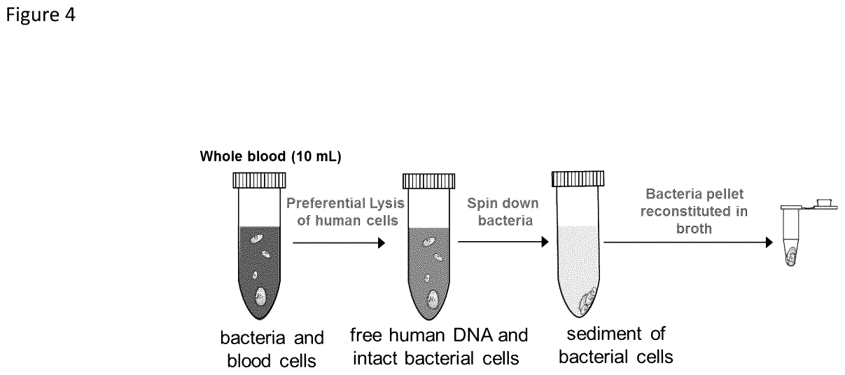

The inventors have designed an assay protocol to enable combined ID and AST into a single step process (FIG. 1) and using the whole blood sample as an example (FIG. 4). In one embodiment is a protocol designed to facilitate direct implementation of singe-cell diagnostics on the dual-digital array chip described below. This protocol bases on a single-tube assay using qPCR/HRMA to detect the bacterial growth in varying drug conditions and identify the bacteria species (FIG. 1). While this (bulk) assay may be limited in sensitivity and unable to resolve mixed species in a single sample, it serves as proof-of-concept of rapid diagnostics by combined ID and AST. The subsequent translation to the microfluidic single-cell assay in described below can facilitate digital diagnostics with enhanced sensitivity, specificity and quantification capability.

Isolation of Intact Bacteria from a Large Volume Blood Sample.

Separating bacterial cells from blood cells and background matrix constituents is critical to detection accuracy. Several existing methods have been developed to clean up samples by removing human cells and proteins; however, they provide non-viable bacterial cells or circulating bacterial DNA for identification though genetic tests, e.g. PCR. While genotypic AST provides a rapid test, it has limited accuracy since the same resistance to an antibiotic agent may be caused by several different mechanisms. Phenotypic AST enables more accurate profiling of bacterial antibiotic susceptibility by direct measurement of cell growth. In order to perform phenotypic AST, the sample preparation method must be able to isolate intact bacterial cells. To this end, the inventors developed a novel sample preparation method for whole blood samples. In the protocol of the present invention (FIG. 4), blood samples are first treated with lysis buffers, which preferentially lyse blood cells but keep bacterial cells viable. DNase I is then added to digest the DNA released from blood cells, and bacterial cells are pelleted by centrifugation. Next, the pellets are re-suspended with broth medium and briefly incubated with antibiotics. This protocol has been tested for isolation of pathogens from 1 ml whole blood samples spiked with 100 cfu of Escherichia coli (E. coli), Staphylococcus aureus (S. aureus), Pseudomonas aeruginosa (P. aeruginosa), and Enterococcus faecalis (E. faecalis), resulting in an encouraging recovery (40-85%) of pathogen cells whose viability was validated through direct culture.

### Comprehensive Diagnostic Assay of Integrated Broad-Scale ID and AST

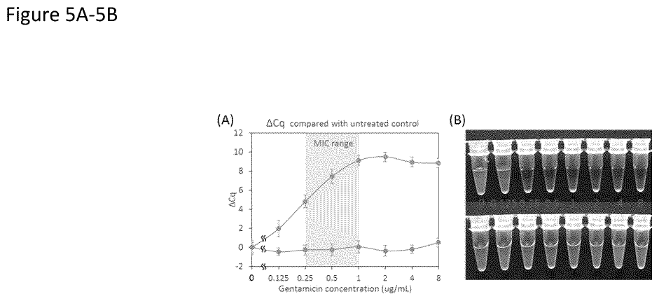

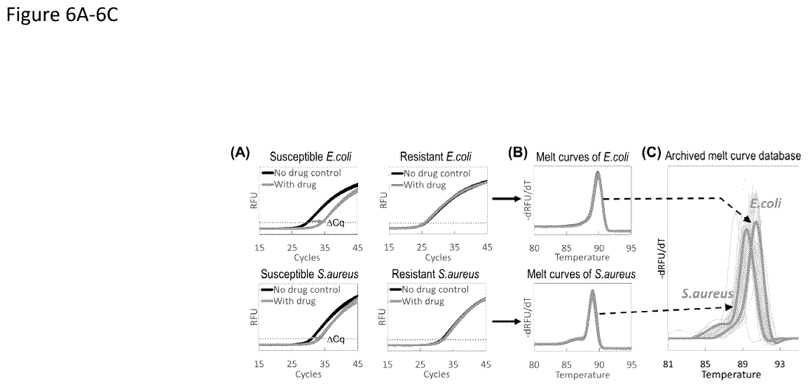

The present invention measures bacterial growth by quantifying the bacterial DNA content using qPCR. As proof of concept, the inventors have expanded the use of their previously developed universal primers for PCR² to demonstrate the correlation of the bacterial 16S rDNA quantity and the bacterial growth (FIG. 1). In this assay, the inventors adopted and modified a thermal lysis protocol that avoids the use of enzymatic or chaotropic reagents that may inhibit PCR. When performing AST, the sample from culture tubes are added to PCR mixes immediately after brief bacterial culture for subsequent detection and quantification of bacterial DNA via qPCR. The quantification cycle (Cq) value indicates the difference in growth rate of different strains under the drug treatment; thereby the antibiotic susceptibility can be determined. This approach was tested using a panel of ESKAPE strains challenged with 5 drugs at 4 concentrations and compare minimum inhibitory concentration (MIC) to conventional broth microdilutional method. FIG. 5 demonstrates that the proposed approach provided MIC range which corresponds to standard CLSI MIC value and the conventional method. A proof-of-concept experiment was conducted to demonstrate the feasibility of rapid phenotypic AST by quantitative detection of bacterial DNA content. As shown in FIG. 6A, after 2 hours of incubation with gentamicin, sensitive strands of both E. coli and S. aureus were measured with an increased Cq value compared to the no-drug (growth) controls. In contrast, resistant strands were measured with a Cq valuable close to the no-drug controls, indicating the growth of bacteria.

While the typical bacterial doubling time for most of the pathogen ranges from 20 to 60 min, the actual time required for reliable detection of bacterial growth can be longer and is dependent on the sensitivity and resolution of the detection method. FIG. 6A shows the detection of bacterial growth within 2 hour of culture by bulk qPCR. With the proposed digital diagnostics platform to be developed in this invention, the bacterial culture and growth detection will be performed via single-cell PCR in pL-sized wells. The inventors expect the required detection time to be shortened due to the superior sensitivity and quantification resolution of this single-cell detection.

### Broad-Scale Pathogen Identification Based on HRMA.

The inventors developed a one-vs-one support vector machine (SVM) algorithm for pathogen identification based on HRMA of the 16S rDNA gene. This technique was implemented for broad-scale pathogen identification after the measurement of bacterial growth. After qPCR, 5 min of temperature ramping was conducted to generate a melt curve, which can be matched with that in the database to determine the target pathogen. The inventors were able to correctly identify the target pathogens (E. coli and S. aureus) by matching the measured melt curves to the previously developed database using the SVM algorithm² (FIG. 6C).

Integrated Broad-Scale ID and AST with High Resolution can be Achieved with Digital PCR (dPCR) for Genetic Analysis of Pathogen DNA.

Despite the promise of the board scale detection and rapid AST, several limitations remain problematic with the bulk qPCR/HRMA assay. Firstly, qPCR in bulk solution has limited quantification resolution, especially when analyzing low concentration pathogens, and thus it may not be able to reliably detect bacterial growth based on the Cq value. Secondly, polymicrobial or contaminated samples may be misidentified or may give erroneous errors due to the confounded melt curve measured in bulk. These problems can be resolved through digital analysis where each single molecule of bacterial DNA is individually assessed by digital PCR (dPCR) and digital HRMA (dHRMA).

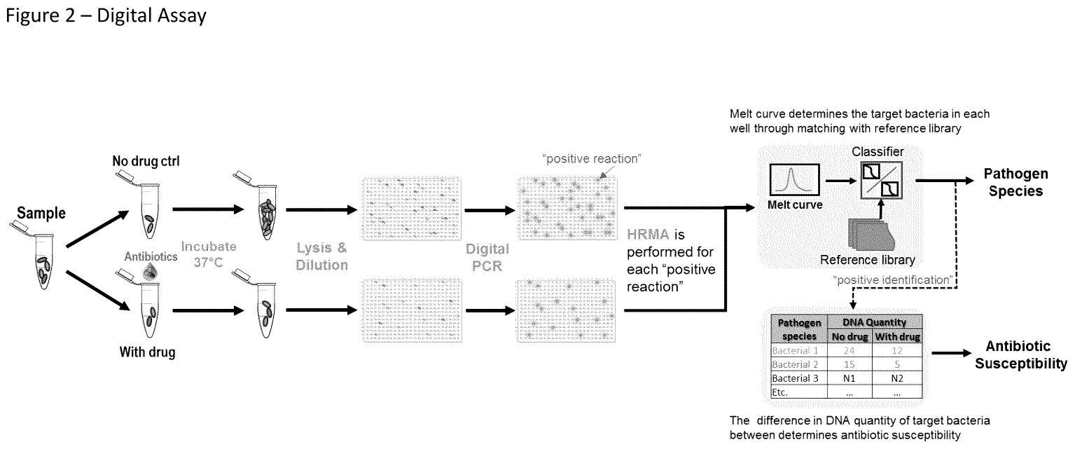

This method (FIG. 2) begins by splitting the sample to incubate with and without antibiotic. Following bacterial cell lysis, the lysate or extracted DNA is mixed with PCR mixture, which comprises of a fluorescent intercalation dye. The mixture is then divided into a large number of reaction chambers so that there is either zero or one bacterial DNA molecule presented in each chamber to perform dPCR and dHRMA. After the reactions, the reaction chamber containing bacterial DNA shows a bright fluorescent level and is called a “positive reaction”, while the reaction chamber without any target DNA exhibits only the background fluorescence. The species of unidentified bacteria in each positive reactions is then determined preferably using HRMA.

The species of unidentified bacteria is identified by determining a melting curve of PCR amplicons in each chamber and comparing it to the database of melting curves of PCR amplicons of known bacteria stored in a computer wherein a “positive identification” occurs when the first melting curve of the unidentified bacteria is equivalent to the one or more melting curves of known bacteria stored in the computer.

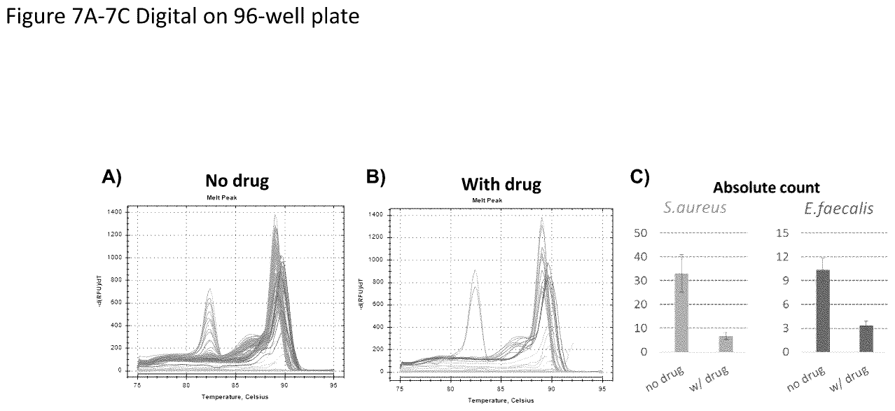

The total number of DNA molecules of each species is then quantified by counting the number of reaction chambers of “positive identification”. Each species will be determined to be antibiotic resistant or sensitive by comparing the number of DNA molecules between the bacteria grown in a broth having an antibiotic and the same bacteria grown in a broth without an antibiotic. The inventors have conducted a proof-of-concept experiment by mixing sensitive strains of S. aureus and E. faecalis and incubating them for 2 hours with and without 8 μg/mL of gentamicin. Bacterial DNA was extracted and diluted 60000-fold such that either 0 or 1 copy of DNA was loaded into each well of a 96-well microtiter plate. Following PCR and HRMA, species identification was performed on the melting curves from both with and without gentamicin-treated plates. The counted of both species with no-gentamicin controls were greater than gentamicin-treated samples by approximately 3-5 folds indicating that gentamicin had effectively inhibited the growth of both bacteria (FIG. 7).

By investigating a single molecule of bacterial DNA, heterogeneous sub-populations can be differentiated and quantified, which enables combined ID/AST assay for coinfection detection and also allows for distinguishing whether a detected molecule is a true pathogen or contaminant in instances where it could be either. Furthermore, counting individual molecules improves detection sensitivity, which can potentially reduce the required incubation time in order to detect bacterial growth.

Integrated ID and AST with Single-Cell Resolution can be Achieved with a Large Number of Digital (Single-Cell) Reaction Chambers for Culture, and Genetic Analysis of Pathogens.

This combined ID/AST assay can be further implemented to perform single-cell analysis where an array of digital reaction chambers is used for identifying and quantifying the growth of individual cells. Encapsulating a single cell in the small volume leads to confinement of bacterial DNA of a high concentration. The high DNA concentration facilitates unambiguous detection of bacterial replications to determine the antibiotic susceptibility. Furthermore, since the melt curve is measured at the single-cell resolution, heterogeneous sub-populations can be differentiated and quantified. Additionally, the single-cell analysis enables “absolute quantification” of bacterial load without the need for calibration.

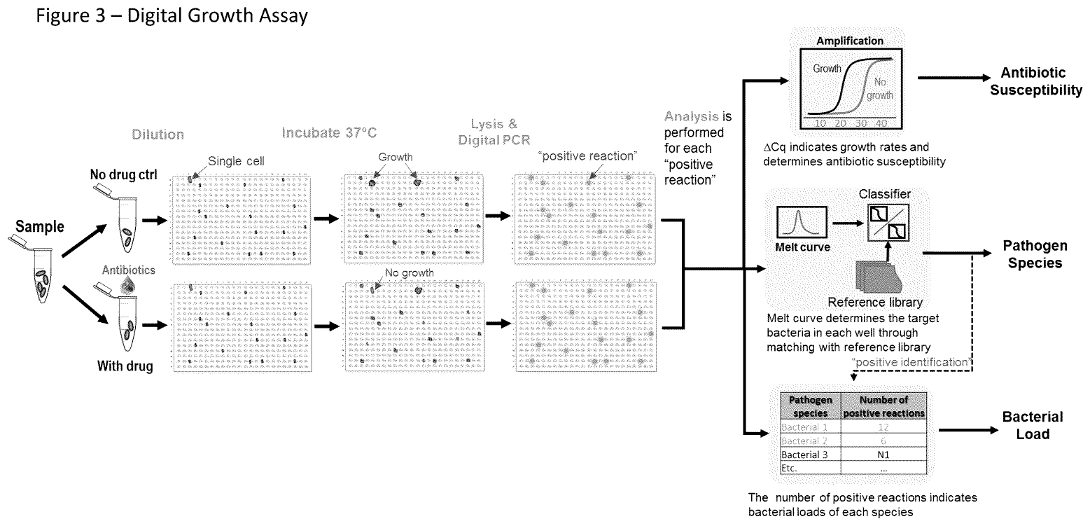

The method (FIG. 3) starts by dividing the samples into 2 portions and mixed with broth in the presence and absence of antibiotic before digital dilution across a large number of reaction chambers. The sample is diluted such that there is either one or zero bacterial cell in each chamber. Then, isolated bacterial cells are briefly grown followed by performing single-cell PCR and HRMA. Cell lysis and DNA extraction step may be performed prior to or together with the PCR step. After PCR, a chamber containing a bacterial cell shows a bright fluorescent signal and is called a “positive reaction”, while the reaction chambers without any bacteria exhibits only the background fluorescence. The species of unidentified bacteria in each positive reaction is first determined preferably using HRMA.

The species of unidentified bacteria is identified by determining a melting curve of PCR amplicons in each chamber and comparing it to the database of melting curves of PCR amplicons of known bacteria stored in a computer wherein a “positive identification” occurs when the first melting curve of the unidentified bacteria is equivalent to the one or more melting curves of known bacteria stored in the computer.

The bacterial load of each species is quantified by counting the number of reaction chamber of “positive identification”.

Importantly, Cq of PCR in each chamber represents the quantity of bacterial DNA, which relates to growth of each bacterial cell. Therefore, each bacterial species will be determined to be antibiotic resistant or sensitive by comparing the average Cq number of the reaction chambers of “positive identification” corresponding to the species between the bacteria grown in a broth having an antibiotic and the same bacteria grown in a broth without an antibiotic.

Integrated ID and AST with Single-Cell Resolution can be Achieved with a Novel Microfluidic Chip for Digitization, Culture, and Genetic Analysis of Pathogens.

To facilitate ID/AST assay at single cell resolution, the inventors have conceptually developed a novel microfluidic chip that realizes this concept of digital assay. The inventors will demonstrate rapid and massively parallelized assessment of single-cell culture, growth measurement via qPCR for quantification of bacterial DNA and strain identification via HRMA.

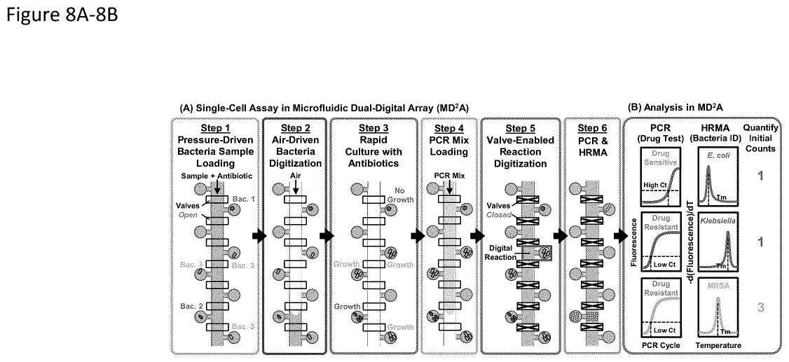

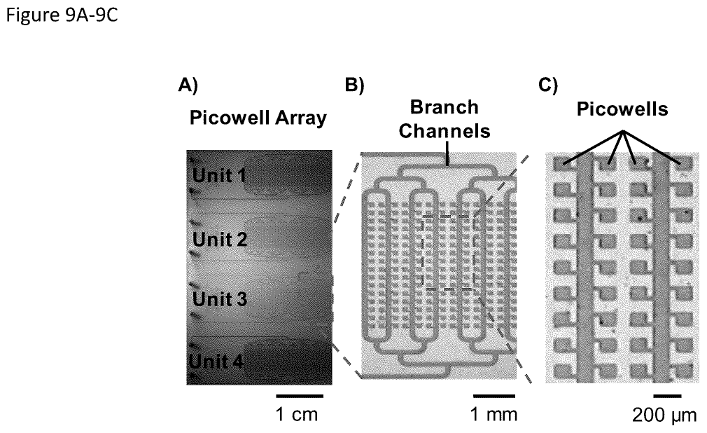

The inventors have developed conceptually a novel Microfluidic Dual-Digital Array (MD²A) chip that utilizes a facile approach to integrate bacteria loading, digitization, culture, reagent switching, PCR, and melt curve analysis in a monolithic device (FIG. 6, FIG. 8 step 1). This chip will enable single-cell analysis with low background, high sensitivity, and wide dynamic range (up to 5-6 orders of magnitude of dynamic range). An enabling feature of the MD²A chip is its dual-level partitioning mechanisms that first isolates single bacteria culturing and then performs digital genetic analysis. Specifically, after bacteria sample and antibiotics are loaded into the chip and each picowell contains either 0 or 1 bacterial cell (based on Poisson distribution), air is used to isolate single cells in the picowells and achieve single-cell culturing (FIG. 8A, steps 1-3). After a brief culturing period, a mixture of lysis buffer and PCR mix is injected. Microvalves are then pressurized to enclose a segment of the channel around each picowell, forming an isolated reaction chamber for PCR amplification and genetic analysis (FIG. 8A, steps 4-6). Critically, the volume of each digital reaction chamber is ˜20-fold greater than the picowell, which allows PCR to work without additional processing steps. Our MD²A chip enables three analyses for bacteria identification, drug resistance profiling, and quantification (FIG. 8B). First, quantitative PCR (qPCR) of the previously described 16S assay will be conducted to determine the bacterial DNA quantity in each picowell. As such, bacterial growth is directly determined by DNA quantity. Bacterial drug resistance results in a low Cq valuate (high DNA quantity) similar to that of no-drug control. In contrast, drug susceptibility results in a high Cq value (low DNA quantity). Post-PCR high resolution melt analysis (HRMA) in each digital reaction chamber will enable identification of multiple pathogens (polymicrobial infection) and differentiation of infected pathogens from the contamination or flora. Initial bacterial load of the sample can also be quantified by counting the number of amplified PCR chambers. Finally, MD²A chip will also be partitioned into multiple units to allow cell culture under various drug conditions (FIG. 9).

### Fabrication of MD²A Chip.

The main material for the MD²A chip will be polydimethylsiloxane (PDMS). The device is composed of a fluidic channel and picowell layer and a microvalve network layer. The fabrication of a device with these layers will be achieved using a modified multilayer soft lithography technique as we have demonstrated in the past (H. Zec, C. O'Keefe, P. Ma and T. H. Wang, “Ultra-thin, evaporation-resistent PDMS devices for absolute quantification of DNA using digital PCR,” 2015 Transducers-2015 18th International Conference on Solid-State Sensors, Actuators and Microsystems (TRANSDUCERS), Anchorage, AK, 2015, pp. 536-539. doi: 10.1109/TRANSDUCERS.2015.7180979). Briefly, molds for the fluid layer and the microvalve layer will be microfabricated on separate silicon wafers via photolithography. Replicas of these molds are then generated using PDMS casting on the molds. The fluidic and microvalve layer replicas are then aligned and thermally bonded in an oven to generate a single chip. Holes are punched in this chip at input and output ports for making fluidic connections with the chip. Finally the bottom surface of the device (containing the microvalve layer) is bonded to a cover glass, readying the chip for experiments.

In one embodiment of the present invention the inventors have designed and fabricated an array chip that contains four identical units, each housing 960 picowells (250 pL in volume) that are connected through inlet/outlet branching channels (FIG. 9). The four-module design allows us to simultaneously test 3 antibiotics and include a no-antibiotic control per chip.

### Sample Loading, Digitization, and Reagent Exchange.

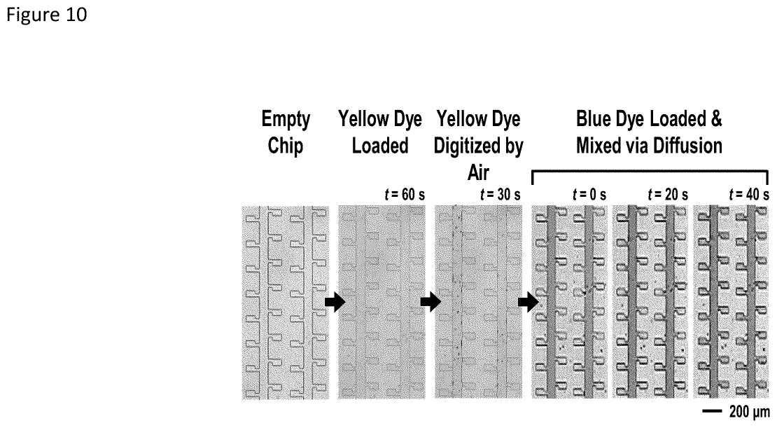

The present invention provides a robust process for sample loading, digitization, and reagent exchange (FIG. 10), which is enabled by the PDMS material, as one example, chosen for fabricating the chip. Sample will be loaded by air pressure while the outlet holes of the chip are sealed with tape. Because PDMS is air permeable, samples (represented by yellow dye in FIG. 10) injected into the device via pressure (e.g., 10 psi) will displace the air in channels and picowells as air escapes through PDMS. Subsequently, with the outlet seal removed, a column of air is injected via low pressure (e.g., 1 psi) into the device. This pressurized air will flow through the channels to the outlet but not into picowells, which are filled with samples, thereby achieving effective digitization within minutes. In addition to sample digitization, air in the channels also provides a good environment for bacteria culturing. Following a brief incubation period (i.e., for bacteria culturing), PCR mix (represented by blue dye in FIG. 10) will be injected into the device via low pressure (e.g., 1 psi). Because the channel is previously filled with only air, PCR mix will easily flow through the channels to achieve reagent switching. Critically, PCR mix will not flow into picowells, as they are occupied by samples. The small volume within the device facilitates rapid diffusion and reagent exchange. For example, the blue dye injected into the channels appears to have mixed with the yellow dye in the picowells after only 40 s (FIG. 10). In addition to simplicity and robustness, this pressure-driven loading and air-driven digitization process simplifies device preparation by obviating hydrophobic surface treatments.

### MD²A Assay and Data Analysis.

One embodiment of MD²A chips and assays of the present invention include chips that house ˜1000 picowells/digital chambers. S. aureus will first be used as the model bacteria. Fixed input S. aureus concentration (e.g., 1×106 CFU/mL) and digitally culturing S. aureus cells will be used with a fixed culturing time (e.g., 2 hours), and finally PCR mix will be injected and PCR and HRMA performed using the PCR reagents and primers disclosed. Using the inventors' custom-built optical detection instrument, the inventors are able to acquire fluorescent images of the MD²A chip at each PCR cycle, as well as at each temperature point during the melt curve experiment. Custom MATLAB programs are being developed to measure the fluorescence intensities within each digital chamber over the course of PCR and melt curve experiment.

A data analysis protocol for PCR will be formed by first plot fluorescence intensity-based real-time PCR curves for all isolated (digital) chambers and measure the Cq values for the chambers with amplified PCR products. The total number of chambers with high fluorescence intensities at the end of PCR allows the inventors to count the initial concentration of S. aureus. A data analysis protocol for HRMA, will be formed by focusing on digital chambers with amplified PCR products and plot the melt curves and acquire the melt signature (e.g., peak temperature values or peak heights) of S. aureus. For PCR and HRMA results, the inventors can average the Cq values and melt curves across the entire MD²A chip. Alternatively, the inventors can also plot the Cq values and melt signatures into histograms for statistical analysis and understanding the population distribution.

After establishing a baseline analyses, the inventors will vary the input S. aureus concentrations, the culturing time, and will also add antibiotics (e.g., penicillin) to the sample. With the changes to the input S. aureus concentrations, the inventors will count different numbers of digital chambers with high fluorescence intensities and they will verify that they are able to still correctly measure the number of digital chambers in accordance to Poisson distribution. As the culturing time increases, the inventors will measure decreased average Cq value from the MD²A chip after PCR. Finally, with the addition of a high concentration of antibiotic that will inhibit S. aureus growth, the inventors will measure a high average Cq value from the MD²A chip after PCR.

### Optimization of MD²A Chip and Assay.

To analyze single cells with a wide dynamic range and test numerous antibiotic conditions, MD²A chip will harbor a massive number of picowells/digital chambers. The number and volume of picowells will also support the input sample volume (˜20-50 μL) from concentrated cells prepared by the upstream blood processing methods. The number of picowells could be in the range of thousands to millions of wells. Furthermore, the chip will be divided into multiple units for AST at different drug conditions. For example, the design of 25 units will allow testing of 6 drugs at 4 concentrations in addition to a no-drug control. On the other hand, the massive array of picowells/digital chambers still must fit within the imaging area of the optical detection instrument (32.9 by 43.8 mm). Therefore, we will extensively optimize the MD²A chip and assay.

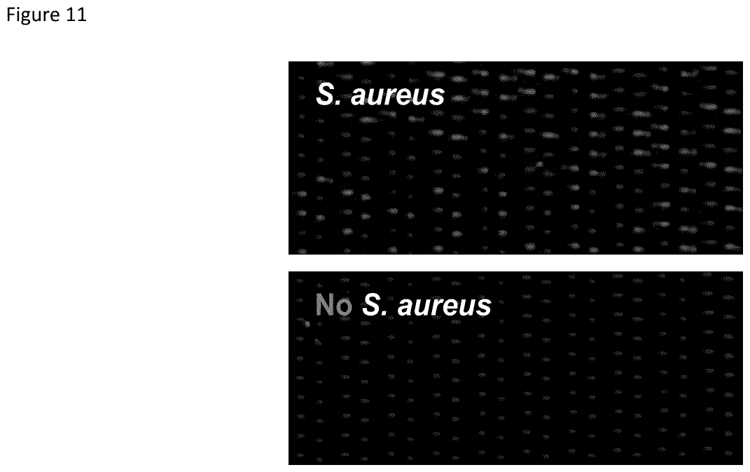

Specifically, the inventors will optimize the volumes the picowells and the digital reaction chambers, as well as the dimensions for the microvalves and pitches. The inventors will also optimize the conditions of their digital PCR process. We will continue working with S. aureus in these optimization experiments. We will initially design 25-pL picowells (e.g., 50 μm (L) by 20 μm (W) by 25 μm (H)), isolate single S. aureus cells (equivalent to 4×10⁷ cells/mL), and verify that these picowells still retain sufficient culture broth and will support S. aureus growth. The digital reaction chambers will be 500 pL (e.g., 100 μm (L) by 200 μm (W) by 25 μm (H), or 20-fold greater than the picowells in volume. The results demonstrate that single S. aureus cell PCR can be reliably achieved at such volume (FIG. 11). Of note, because S. aureus is a Gram-positive bacterium, the results also clearly demonstrate the effectiveness of our lysis buffer. Additional benchtop experiments may be performed to determine if the volumetric ratio between the culture broth and the PCR can be increased, which may allow one to further reduce the volume of the digital chambers. On the other hand, the lower limit of the digital chamber dimensions may be dictated by the sensitivity of the optical detection instrument. To ensure high fluorescence signals from PCR, one may optimize the conditions of digital PCR by adjusting the concentrations of the polymerase, primers, additives (e.g., BSA and Tween20), as well as the annealing temperature. One may also minimize the pitches between picowells until adjacent wells cannot be resolved by the optical detection instrument. Finally, one may reduce the length, width, and membrane thickness of microvalves. The typical dimensions for the microvalves of the present invention are 200 μm (L) by 200 μm (W), as one example. One may reduce the lengths and widths while adjusting the micro valve membrane thickness to ensure proper closure of microvalves and digitization of PCR.

Validation with Multiple Bacteria and Antibiotics.

To validate our MD²A chip and assay, the inventors will detect and quantify 4 reference strains of bacteria and measure their resistance to 6 representative antibiotics. Following the CLSI guideline, the inventors will culture E. coli with ampicillin and gentamicin, S. aureus with penicillin and trimethoprim-sulfamethoxazole, P. aeruginosa with ceftazidime and gentamicin, and E. faecalis with ampicillin and penicillin. For each pair of bacteria and antibiotic, the inventors will perform a no-antibiotic control and test a high antibiotic concentration that will inhibit bacterial growth. In these experiments, the no-antibiotic control will yield low Cq values, whereas the high antibiotic concentration samples will yield high Cq values, indicating growth inhibition due to the antibiotics. Subsequent dHRMA will verify the identity of each strain. The inventors will also vary the initial sample concentrations for each bacterial strain and confirm that our MD²A chip and assay will indeed quantify the correct input concentration. Finally, to demonstrate that MD²A chip has the capacity to detect polymicrobial infections, the inventors will mix two strains and perform the assay.

### Chip Assembly

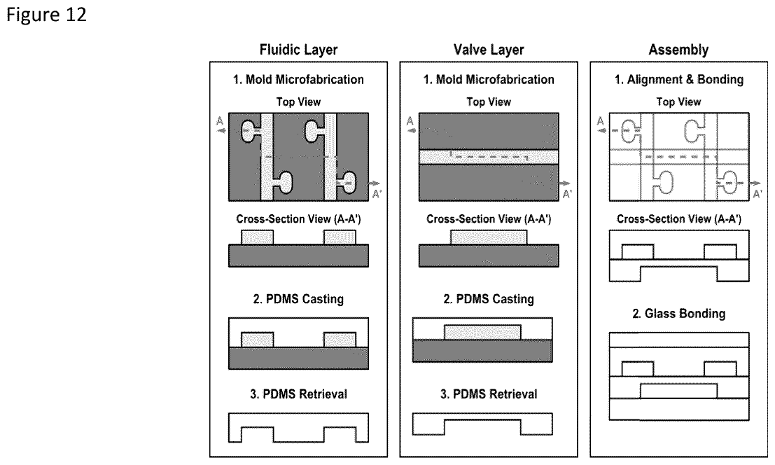

FIG. 12 provides one embodiment of chip assembly. Briefly, molds for the fluid layer and the microvalve layer will be microfabricated on separate silicon wafers via photolithography. Replicas of these molds are then generated using PDMS casting on the molds. The fluidic and microvalve layer replicas are then aligned and thermally bonded in an oven to generate a single chip. Holes are punched in this chip at input and output ports for making fluidic connections with the chip. Finally, both the top and bottom surfaces of the chip are bonded to coverglasses, readying the chip for experiments.

### Chip System

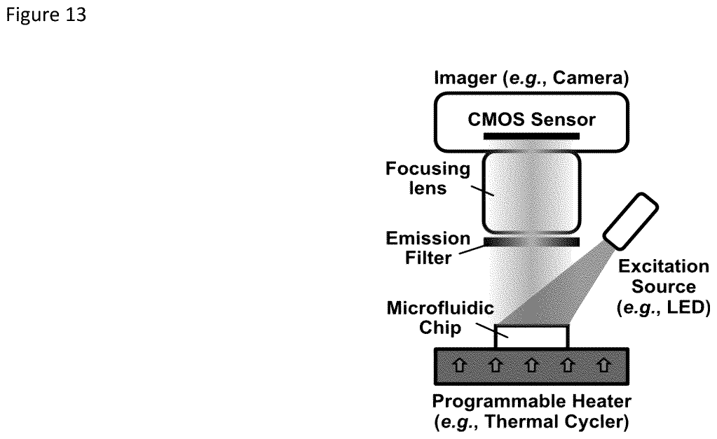

FIG. 13 provides one embodiment of a chip system. As observed, a chip system of the present invention includes a wide-field, high-sensitivity, real-time fluorescence imaging device that can image the entire microfluidic chip (FIG. 9A) and a programmable heater. The imaging device is composed of an excitation source (e.g., LED), an appropriate emission filter, and a detector (e.g., a high resolution CMOS digital camera). The chip is placed on the surface of the programmable heater, which is responsible for thermal cycling for PCR and HRMA.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for identifying and quantifying the growth of antibiotic resistant or sensitive bacteria comprising the steps of:
culturing a first amount of an unidentified bacteria from a biological sample obtained from a subject in a first broth comprising an antibiotic;
culturing a second amount of the unidentified bacteria from the biological sample obtained from the subject in a second broth substantially free of the antibiotic, wherein the first and second amounts are identical to one another;
lysing the unidentified bacteria in the first and second broth to release bacteria nucleic acid;
diluting the bacteria nucleic acids of unidentified bacteria in first and the second broth and then placing them in two separate arrays of wells so that each well contains no more than one copy of the bacteria nucleic acid;
amplifying the bacteria nucleic acid in each well with a mixture including a fluorescent intercalating dye using polymerase chain reaction (PCR) forming amplified DNA; and
identifying a species of the unidentified bacteria or an identified bacteria species by determining a first melting curve of the amplified DNA in each well and comparing it to one or more melting curves of PCR amplicons of known bacteria stored in a computer in which a positive identification occurs when the first melting curve of the amplified DNA in the well is equivalent to the one or more melting curves of the PCR amplicons stored in the computer; and identifying the growth of each of the identified bacteria species by determining the amount of bacteria nucleic acid of the identified bacteria species calculated by the number of the wells of the positive identification associated to the identified bacteria species;
wherein an identified bacterial species is antibiotic resistant based on the difference of the amount of nucleic acids of the bacteria derived from the first broth and the second.

2. The method of claim 1 comprising a step of separating the bacteria from the biological sample.

3. The method of claim 1, wherein the biological sample is selected from the group consisting of whole blood, plasma, serum, RBC fraction, urine, saliva, cerebrospinal fluid, semen, sweat, bile, gastric contents, breast milk, exudates, ascites, lymph, sputum, lavage fluid, bronchial fluid, or a combination thereof.

4. The method of claim 1 wherein the biological sample is blood and the method comprises a step of lysing cells of the blood but not the bacteria.

5. The method of claim 1 wherein the culturing the bacteria is in the range of 15 minutes to 24 hours.

6. The method of claim 1 further comprising the step of identifying the species of unidentified bacteria using high-resolution melt curve analysis (HRMA) to generate the first melting curve of the unidentified bacteria.
